(19) United States
(12) Patent Application Publication
(10) Pub. No.: US 2016/0115532 A1
(43) Pub. Date: Apr. 28, 2016

(54) HIGH SENSITIVITY MUTATION DETECTION USING SEQUENCE TAGS

(71) Applicant: Sequenta, Inc., South San Francisco, CA (US)

(72) Inventor: Malek Faham, South San Francisco, CA (US)

(21) Appl. No.: 14/420,823

(22) PCT Filed: Aug. 8, 2013
(86) PCT No.: PCT/US2013/054189

§ 371 Date: Feb. 10, 2015

Related U.S. Application Data
(60) Provisional application No. 61/682,113, filed on Aug. 10, 2012.

(51) Int. Cl. C12Q 1/68

(52) U.S. Cl. CPC C12Q 1/6874

(57) ABSTRACT

The invention is directed to methods for increasing the sensitivity of high throughput sequencing, particularly for distinguishing true rare mutations from amplification, sequencing and other sample processing errors that occur in sequencing techniques. In one aspect, methods of the invention includes steps of (a) preparing templates from nucleic acids in a sample; (b) labeling by sampling the templates to form tag-template conjugates, wherein substantially every template of a tag-template conjugate has a unique sequence tag; (c) linearly amplifying the tag-template conjugates; (d) generating a plurality of sequence reads from the linearly amplified tag-template conjugates; and (e) determining a nucleotide sequence of each of the nucleic acids based on the frequencies, or numbers, of each type of nucleotide at each nucleotide position of each plurality of sequence reads having identical sequence tags.

### CROSS-REFERENCE

This application claims the benefit of U.S. Provisional Patent Application No. 61/682,113 filed Aug. 10, 2012, which is herein incorporated by reference in its entirety.

### BACKGROUND OF THE INVENTION

The development of high throughput, or next generation, DNA sequencing technologies has revolutionized cancer research by providing tools for measuring with unprecedented resolution the genetic alterations associated with cancers, e.g. Stratton, Science, 331: 1553-1558 (2011); Parmigiani et al, Genomics, 93(1): 17 (2009); Greenman et al, Nature, 446 (7132): 153-158 (2007); Leary et al, Science Translational Medicine, 2(20): 20ra14 (24 Feb. 2010). Although a direct role for these technologies in cancer medicine, e.g. in diagnosis, prognosis and screening, seems imminent, many challenges must be overcome before such applications are realized. For example, the determination of relevant cancer sequences is affected not only by the biology of a cancer, but also by the presence of normal tissue, sample preparation and handling, nucleic acid extraction, amplification techniques, and sequencing chemistries, e.g. Stratton (cited above). In particular, the relatively high level of amplification and sequencing errors makes screening and detection of rare mutations difficult, despite the huge sequencing capacity of next-generation sequencing instruments. This latter challenge has been addressed by several groups with a variety of approaches that include both enhanced data analysis as well as technical modifications to permit detection and tracking of amplification and sequencing errors, e.g. Flaherty et al, Nucleic Acids Research, 40(1): e2 (2012); Campbell et al, Proc. Natl. Acad. Sci., 105: 13081-13086 (2008); Kinde et al, Proc. Natl. Acad. Sci., 108: 9530-9535 (2011); Schmitt et al, Proc. Natl. Acad. Sci., (PNAS Early Edition 1208715109, 2012); and the like.

In view of the importance of accurate detection of rare mutations in cancer, it would be a significant advance in the field if methods were available that overcame the limitations of current high throughput sequencing methodologies in this area.

### SUMMARY OF THE INVENTION

The present invention is directed to methods for using sequence tags to improve the accuracy and sensitivity of detecting rare mutations from high throughput DNA sequencing by providing sequencing templates that have be directly copied from one or both strands of target nucleic acids. The invention is exemplified in a number of implementations and applications, some of which are summarized below and throughout the specification.

The invention includes methods for sequencing nucleic acids to detect rare mutants comprising the following steps: (a) preparing templates from nucleic acids in a sample; (b) labeling by sampling the templates to form tag-template conjugates, wherein substantially every template of a tag-template conjugate has a unique sequence tag; (c) linearly amplifying the tag-template conjugates; (d) generating a plurality of sequence reads for each of the linearly amplified tag-template conjugates; and (e) determining a nucleotide sequence of each of the nucleic acids based on the frequencies, or numbers, of each type of nucleotide at each nucleotide position of each plurality of sequence reads having identical sequence tags. In some embodiments, such step of determining includes determining a plurality nucleotide at each nucleotide position of each of the plurality of sequence reads having identical sequence tags.

These above-characterized aspects, as well as other aspects, of the present invention are exemplified in a number of illustrated implementations and applications, some of which are shown in the figures and characterized in the claims section that follows. However, the above summary is not intended to describe each illustrated embodiment or every implementation of the invention.

### BRIEF DESCRIPTION OF THE DRAWINGS

The novel features of the invention are set forth with particularity in the appended claims. A better understanding of the features and advantages of the present invention is obtained by reference to the following detailed description that sets forth illustrative embodiments, in which the principles of the invention are utilized, and the accompanying drawings of which:

FIGS. 1A-1E illustrate examples of labeling by sampling to attach unique sequence tags to nucleic acid molecules.

FIG. 2 illustrates an embodiment for attaching unique sequence tags to target nucleic acids followed by the formation of DNA circles for carrying out an RCA reaction.

FIG. 3A illustrates the propagation of errors in methods employing exponential amplification of target nucleic acids.

FIG. 3B illustrates the random occurrence of errors in methods employing linear amplification in which template copies are made only from the original target nucleic acid.

### DETAILED DESCRIPTION OF THE INVENTION

The practice of the present invention may employ, unless otherwise indicated, conventional techniques and descriptions of molecular biology (including recombinant techniques), bioinformatics, cell biology, and biochemistry, which are within the skill of the art. Such conventional techniques include, but are not limited to, sampling and analysis of blood cells, nucleic acid sequencing and analysis, and the like. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV); PCR Primer: A Laboratory Manual; and Molecular Cloning: A Laboratory Manual (all from Cold Spring Harbor Laboratory Press); and the like.

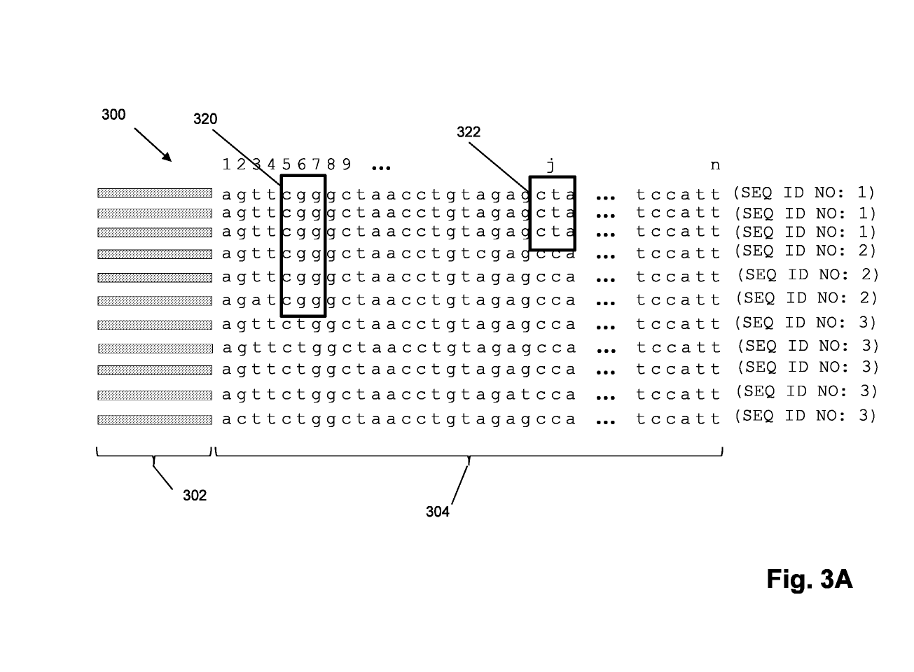

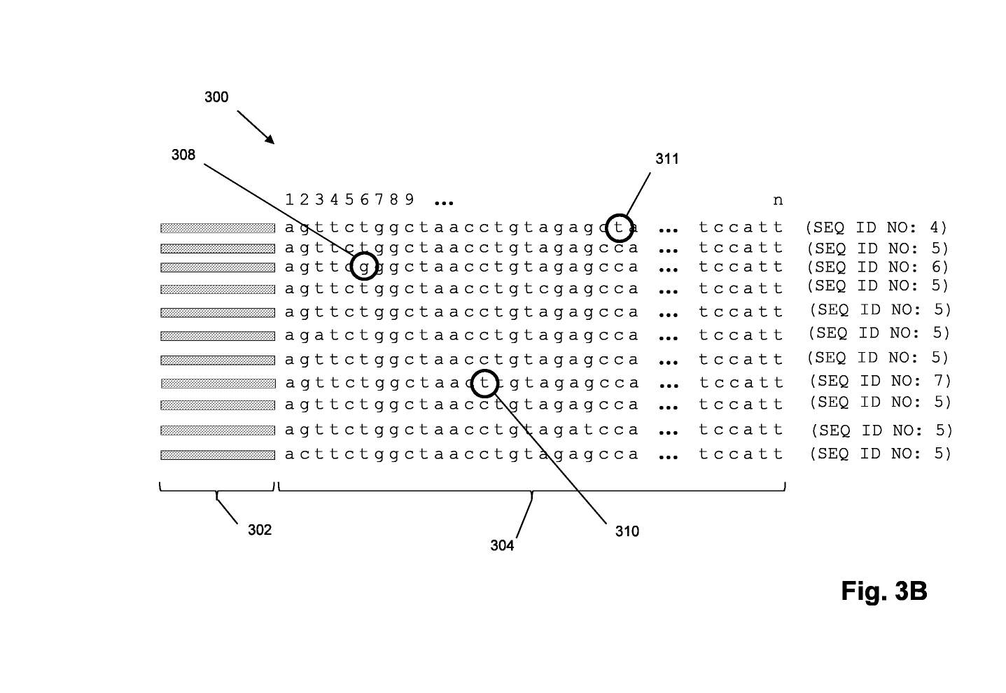

The invention is directed to methods for increasing the sensitivity of high throughput sequencing, particularly for improving techniques for detecting rare mutations. In one aspect, the invention is directed to methods for distinguishing true rare mutations from amplification, sequencing and other sample processing errors that occur in sequencing techniques. In one aspect, methods of the invention employ linear, or non-exponential, amplification of target nucleic acids to produce the copies from which sequence reads are generated. In some embodiments, target nucleic acids are labeled with a unique sequence tag (to form tag-template conjugates) which is copied along with the target nucleic acid. The sequence tag is then used to associate or group all the sequence reads generated from copies originating from the same target nucleic acid. This process overcomes a difficiency in methods employing exponential amplifications where copies are made from copies, thereby permitting errors to accumulate in sequences generated in the later stages of an amplification reaction. FIG. 3A illustrates this point. Sequence tags (302) (which are all the same) associate sequence reads (304) (SEQ ID NO: 1, SEQ ID NO: 2, and SEQ ID NO: 3) in to a group originating from the same tag-template conjugate. As illustrated by the “g's” in column “6” (320) or “t's” in column “j” (322) after an error occurs it is propogated to all subsequently synthesized strands. If the error occurs early on in the amplification reaction, then it may be difficult or impossible to correctly call the true base at a particular location. On the other hand, by maximizing the use of linear amplification, errors are not propagated because each copy is a copy of the original target nucleic acid (or at most a copy of a first copy of the original target nucleic acid). As a result, the pattern of errors is very different, as illustrated in FIG. 3B (which does not take into account sequencing technique-specific biases in errors). If sequence reads (300) (SEQ ID NO: 4, SEQ ID NO: 5, SEQ ID NO: 6, and SEQ ID NO: 7) are aligned as described in FIG. 3A, errors are randomly distributed over the columns and rows of base calls, e.g. (308) (SEQ ID NO: 6), (310) (SEQ ID NO: 7), (311) (SEQ ID NO: 4), and the like. This improves base calling because there are no propagated errors. Base calls of a nucleic acid are made from a plurality of sequence reads in accordance with the invention. In some embodiments, the plurality of sequence reads (comprising separate copies of a tag-template conjugate) is in the range of from about 10 to 1000, or in the range of from about 10 to 100. Base calling from sequence reads aligned by their common tags may be made in a variety of ways. In some embodiments, a base call at a particular position is determined by whatever base is present at such position in the greatest number, or frequency; that is, the plurality nucleotide at that position. Thus, for example, if there are 10 sequence reads and if at given position 4 A's, 2 C's, 3 G's and 1 T are recorded, then the base call at such position would be “A” because it is present in the greatest frequency; that is, it is the plurality nucleotide at that position. In other embodiments, a base call may be made only if a plurality nucleotide is present in a majority of sequence reads at a given position; otherwise, no call would be made. Thus, in the latter embodiment, measurement of 4 A's, 2 C's, 3 G's and 1 T at a position would result in no base call at the position. Other algorithms for base calling based on the frequencies of nucleotides measured at a given position may also be used in the implementation of the invention. Additional algorithms taking into account performance features of particular sequencing chemistries may also be applied (e.g. taking into account base incorporation bias, different signals generated by different labels, sequence context factors such as whether a base is incorporated early or late in a sequence read, and the like).

In one embodiment of the invention, sequence tags are attached to target nucleic acid molecules of a sample by labeling by sampling, e.g. as disclosed by Brenner et al, U.S. Pat. No. 5,846,719; Brenner et al, U.S. Pat. No. 7,537,897; Macevicz, International patent publication WO 2005/111242; and the like, which are incorporated herein by reference. In labeling by sampling, polynucleotides of a population to be labeled (or uniquely tagged) are used to sample (by attachment, linking, or the like) sequence tags of a much larger population. That is, if the population of polynucleotides has K members (including replicates of the same polynucleotide) and the population of sequence tags has N members, then N>>K. In one embodiment, the size of a population of sequence tags used with the invention is at least 10 times the size of the population of clonotypes in a sample; in another embodiment, the size of a population of sequence tags used with the invention is at least 100 times the size of the population of clonotypes in a sample; and in another embodiment, the size of a population of sequence tags used with the invention is at least 1000 times the size of the population of clonotypes in a sample. In other embodiments, a size of sequence tag population is selected so that substantially every clonotype in a sample will have a unique sequence tag whenever such clonotypes are combined with such sequence tag population, e.g. in an attachment reaction, such as a ligation reaction, amplification reaction, or the like. In some embodiments, substantially every clonotype means at least 90 percent of such clonotypes will have a unique sequence tag; in other embodiments, substantially every clonotype means at least 99 percent of such clonotypes will have a unique sequence tag; in other embodiments, substantially every clonotype means at least 99.9 percent of such clonotypes will have a unique sequence tag.

In some embodiments, in which up to 1 million target nucleic acids are labeled by sampling, large sets of sequence tags may be efficiently produced by combinatorial synthesis by reacting a mixture of all four nucleotide precurors at each addition step of a synthesis reaction, e.g. as disclosed in Church, U.S. Pat. No. 5,149,625, which is incorporated by reference. The result is a set of sequence tags having a structure of “N₁N₂ . . . Nₖ” where each Nᵢ=A, C, G or T and k is the number of nucleotides in the sequence tags. The number of sequence tags in a set of sequence tags made by such combinatorial synthesis is 4^{k}. Thus, a set of such sequence tags with k at least 14, or k in the range of about 14 to 18, is appropriate for attaching sequence tags to a 10⁶-member population of molecules by labeling by sampling. Sets of sequence tags with the above structure include many sequences that may introduce difficulties or errors while implementing the methods of the invention. For example, the above combinatorially synthesized set of sequence tags includes many member tags with homopolymers segments that some sequencing approaches, such as sequencing-by-synthesis approaches, have difficulty determining with accuracy above a certain length. Therefore, in some embodiments, the invention includes combinatorially synthesized sequence tags having structures that are efficient for particular method steps, such as sequencing. For example, several sequence tag structures efficient for sequencing-by-synthesis chemistries may be made by dividing the four natural nucleotides into disjoint subsets which are used alternatively in combinatorial synthesis, thereby preventing homopolymer segments above a given length. For example, let z be either A or C and x be either G or T, to give a sequence tag structure of
[(z)₁(z)₂ . . . (z)ᵢ][(x)₁(x)₂ . . . (x)_{J}] . . .
   where i and j, which may be the same or different, are selected to limit the size of any homopolymer segment. In one embodiment, i and j are in the range of from 1 to 6. In other embodiments other pairing of nucleotides may be used, for example, z is A or T and x is G or C; or z is A or G and x is T or C. Alternatively, let z′ be any combination of three of the four natural nucleotides and let x′ be whatever nucleotide is not a z′ (for example, z′ is A, C or G, and x′ is T). This gives a sequence tag structure as follows:
[(z′)₁(z′)₂ . . . (z′)ᵢ]x′[(z′)₁(z′)₂ (z′)ᵢ]x′ . . .
   where i is selected as above and the occurrence of x′ serves as a punctuation to terminate any undesired homopolymers.

A variety of different attachment reactions may be used to attach unique tags to substantially every target nucleic acid in a sample. In one embodiment, such attachment is accomplished by combining a sample containing target nucleic acid molecules with a population or library of sequence tags so that members of the two populations of molecules can randomly combine and become associated or linked, e.g. covalently. In such tag attachment reactions, target nucleic acids may comprise linear single or double stranded polynucleotides and sequence tags are carried by reagent such as amplification primers, such as PCR primers, ligation adaptors, circularizable probes, plasmids, or the like. Several such reagents capable of carrying sequence tag populations are disclosed in Macevicz, U.S. Pat. No. 8,137,936; Faham et al, U.S. Pat. No. 7,862,999; Drmanac et al, U.S. patent publication US 2009/0264299; Zheng et al, U.S. Pat. No. 7,862,999; Landegren et al, U.S. Pat. No. 8,053,188; Unrau and Deugau, Gene, 145: 163-169 (1994); Church, U.S. Pat. No. 5,149,625; and the like, which are incorporated herein by reference.

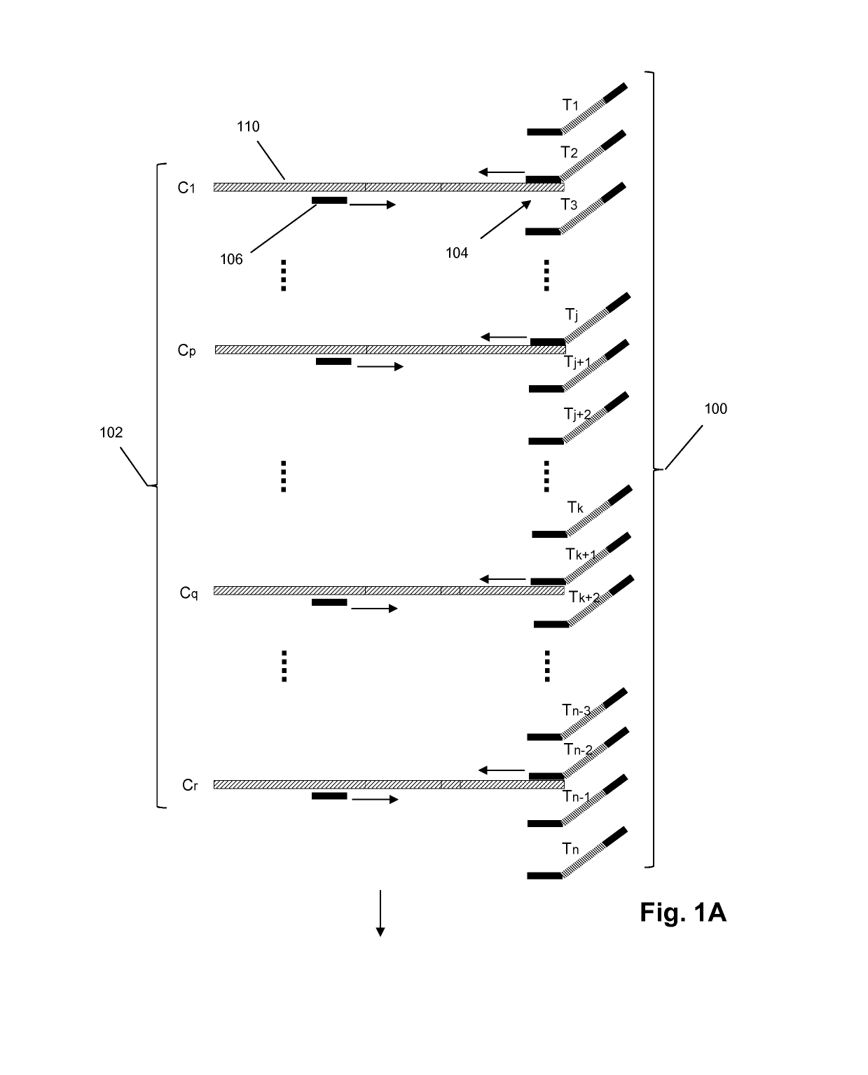

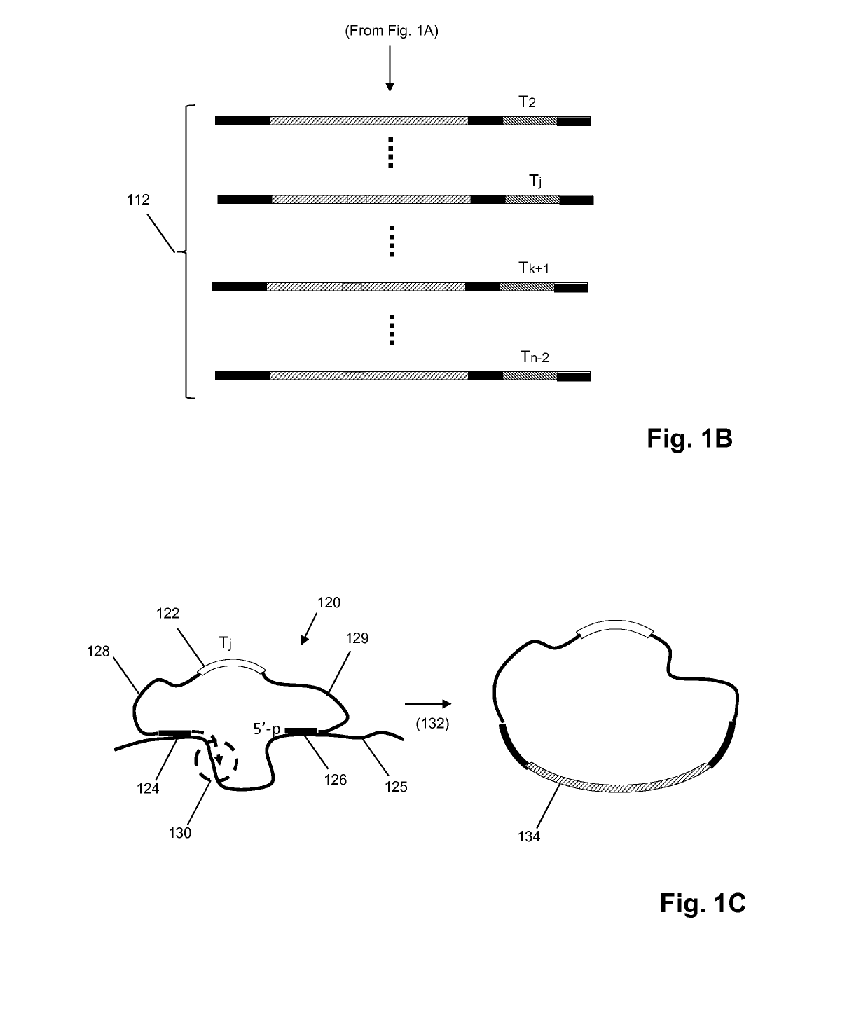

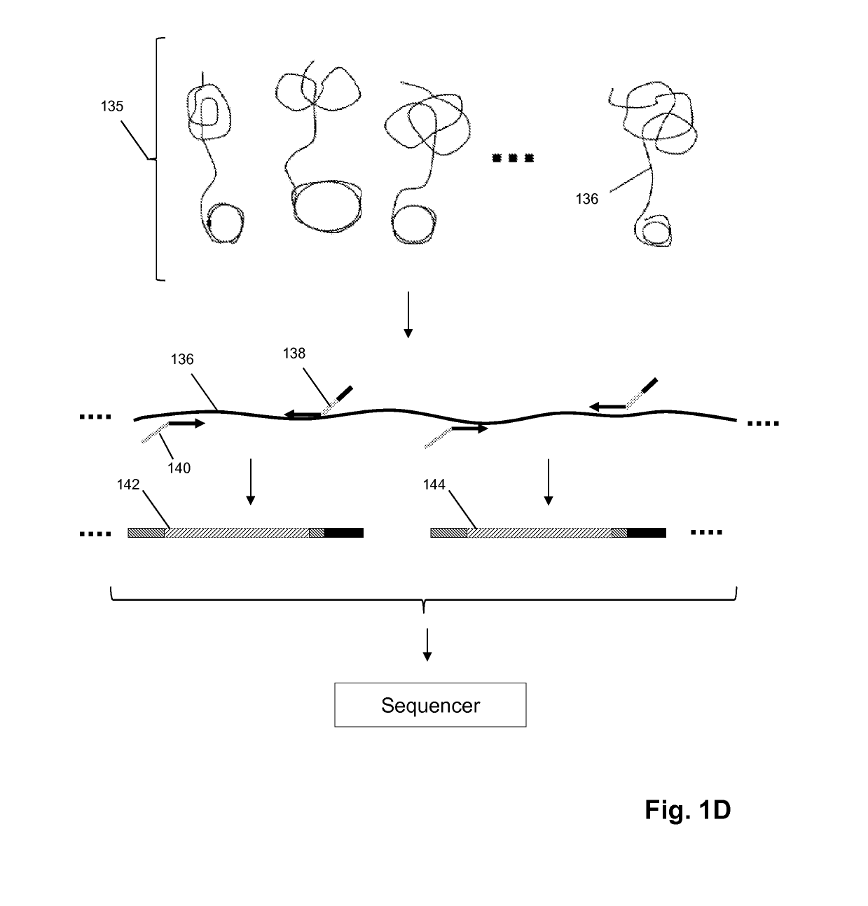

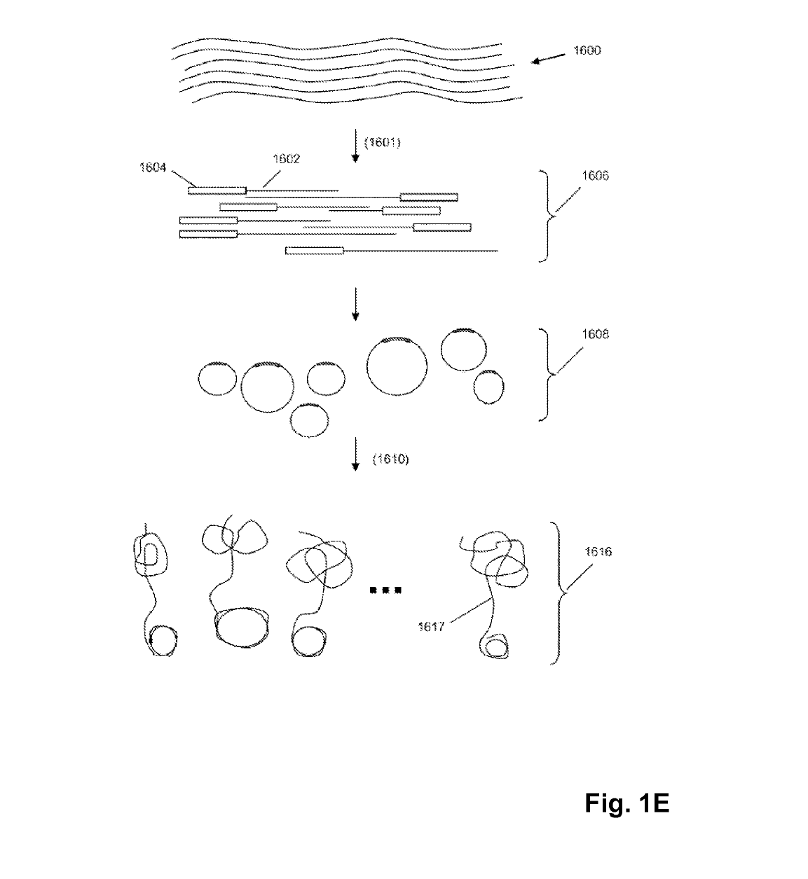

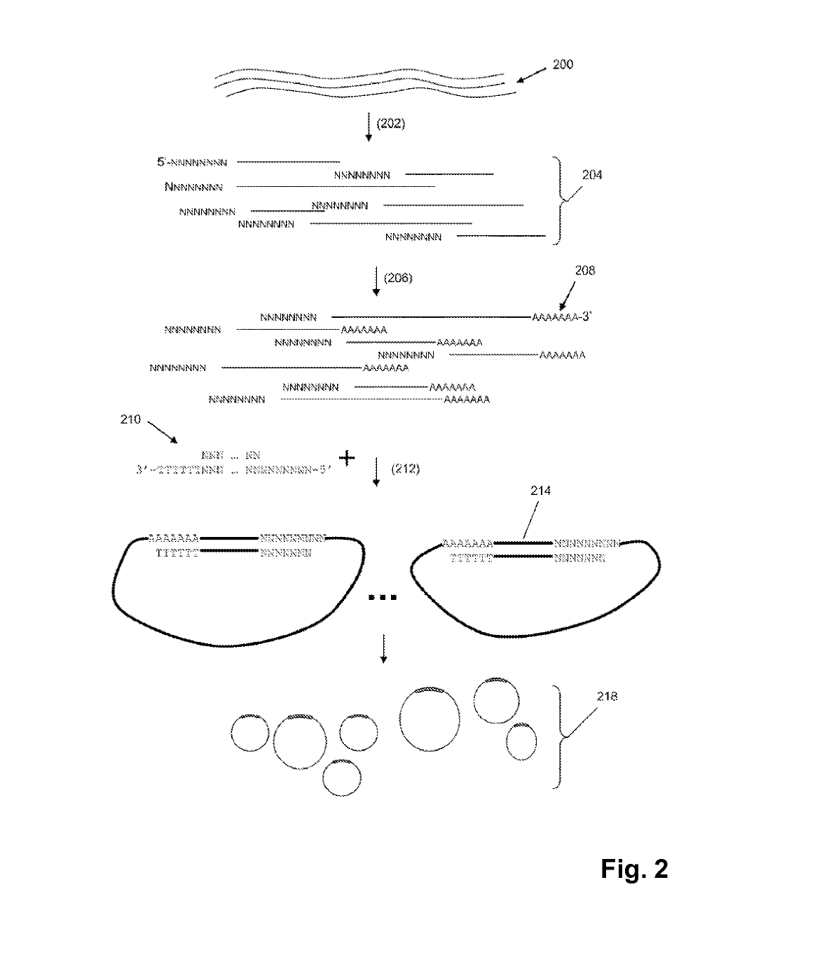

FIGS. 1A and 1B illustrate an attachment reaction comprising a reaction in which a population of sequence tags (T₁, T₂, T₃ . . . Tⱼ, Tⱼ₊₁ . . . Tₖ, Tₖ₊₁ . . . Tₙ₋₁, Tₙ) is incorporated into primers (100) by two or more cycles of annealing and polymerase extension, each separated by a denaturation step. The population of sequence tags has a much greater size than that of target nucleic acid molecules (102). The sequence tags are attached to the target nucleic acid molecules by annealing the primers to the target nucleic acid molecules and extending the primers with a DNA polymerase. The figure depicts how the target nucleic acid molecules select, or sample, a small fraction of the total population of sequence tags by randomly annealing to the primers by way of their common primer binding regions (104). Since the primers (an therefore sequence tags) combine with the target nucleic acid molecules randomly, there is only a small possibility that the same sequence tag may be attached to different nucleic acid molecules; however, if the population of sequence tags is large as taught herein, then such possibility will be negligibly small so that substantially every target nucleic acid molecule will have a unique sequence tag attached. The other primer (106) of the forward and reverse primer pair anneals to another region of the target nucleic acid (110) so that after two or more cycles of annealing, extending and melting, amplicon (112) is formed, thereby attaching unique sequence tags to each target nucleic acid (C₁, . . . Cₚ, . . . C_{q}, . . . and Cᵣ) in population (102). That is, amplicon (112) comprises the tag-template conjugates from the attachment reaction.

FIGS. 1C and 1D illustrate another embodiment for attaching sequence tags by labeling by sampling, after which tag-template conjugates are linearly amplified by RCA. Linear single stranded probe (120) contains sequence tag Tⱼ (122) and first and second target-specific regions (124) and (126) that are capable of specifically hybridizing to separate complementary regions of target nucleic acid (125). First target-specific region (124) has a free 3′ hydroxyl that may be extended by a DNA polymerase (130) in the presence of dNTPs under extension reaction conditions. Usually polymerase (130) lacks chain displacement activity, so that it synthesizes and extension from first target-specific region (124) up to the 5′ end of second target-specific region (126), which has a 5′ phosphate; thus, in the presence of a ligase the extention is ligated to second target-specific region (126) to form (132) closed single stranded circle (133) under ligase reaction conditions. Closed single stranded circle (133) contains a copy (134) of a target nucleic acid and sequence tag (122); that is, it is one embodiment of a tag-template conjugate. Other regions (128) and (129) of probe (120) may contain elements, such as, primer binding sites, endonuclease recognition sites, nickase sites, and the like, for use in later replication and generation of templates. As illustrated in FIG. 1D, circles (135) comprising tag-template conjugates may be replicated in an RCA reaction, after which forward (138) and reverse (140) may be added under conditions permitting them to anneal to primer binding sites flanking the tag-template conjugates in individual strands (136) of the RCA amplicon. After two or more cycles of annealing and extension separated by at least one step of denaturation, tag-template conjugates (142), (144) and the like, are formed that are ready for sequencing, e.g. by an Illumina GA DNA sequencer.

As mentioned above, in some embodiments, the method of the invention may be implemented with the following steps: (a) preparing templates from nucleic acids in a sample; (b) labeling by sampling the templates to form tag-template conjugates, wherein substantially every template of a tag-template conjugate has a unique sequence tag; (c) linearly amplifying the tag-template conjugates; (d) generating a plurality of sequence reads for each of the linearly amplified tag-template conjugates; and (e) determining a nucleotide sequence of each of the nucleic acids based on the frequencies, or numbers, of each type of nucleotide at each nucleotide position of each plurality of sequence reads having identical sequence tags. Templates may be any nucleic acid whose sequence can be determined using a sequencing chemistry and/or approach. Templates may comprise, RNA, single stranded DNA or double stranded DNA. Templates may also comprise transcripts of any of the foregoing that have been modified, for example, by substitution of nucleoside or nucleotide analogs, attachment of labels, such as fluorescent labels, or the like. In some embodiments, after linear amplifying tag-template conjugates to form a first amplicon, member tag-template conjugates of the first amplicon may be further amplified either by a successive linear amplification or an exponential amplification from which sequence reads are determined. For example, in one embodiment, after a first amplicon is formed, member tag-template conjugates are prepared from sequencing by the Illumina sequencing chemistry (e.g. U.S. Pat. Nos. 7,741,463; 8,192,930; 8,158,346; and the like, which are incorporated herein by reference). That is, adaptor sequences are attached to each end of each member tag-template conjugate, wherein the adaptors comprise primer binding sites for bridge amplification on a solid substrate. Typically, in such embodiments, tag-template conjugates are double stranded DNA and double stranded adaptor oligonucleotides are attached by ligation. After disposing the modified tag-template conjugates on such a solid substrate, a bridge amplification reaction is carried out to form second amplicons, or clusters, corresponding to each of the tag-template conjugates from the first amplicon (or a sample thereof). In other sequencing approaches, other secondary amplification schemes may be employed. For example, some sequencing chemistries, such as, pyrosequencing (e.g. as commercialized by 454 Life Sciences) or pH-based sequencing (e.g. as commercialized by Life Technologies), member tag-template conjugates of the first amplicon may be subsequently amplified exponentially by emulsion PCR, e.g. U.S. Pat. Nos. 8,012,690; 7,842,457; U.S. patent publication 2011/0195459; 2011/0195252; and the like, which are incorporated herein by reference). Thus, the step of linearly amplifying the tag-template conjugates to form a first amplicon may be followed by the step of generating a plurality of sequence reads comprising the following steps: (i) amplifying each of the tag-template conjugates to form second amplicons for each of such tag-template conjugate, and (ii) determining the nucleotide sequence of each of the tag-template conjugates in each of the second amplicons to provide a sequence read for each second amplicon. In some embodiments, the step of amplifying may be carried out by bridge PCR. In other embodiments, the step of amplifying may be carried out by emulsion PCR.

### Circularization of Target Nucleic Acids

In some embodiments of the invention, a method for attaching sequence tags to a target nucleic acid, e.g. a fragment of genomic DNA, begins with ligation of a first adaptor (containing a sequence tag) followed by circle formation. Genomic fragments of 100 to 300 (or 300-600) bases in length may be prepared by DNAse fragmentation that generates 5-prime phosphates and 3-prime OH groups suitable for ligation. High-complexity genomic DNA can be prepared as single stranded (ss) DNA by heating (denaturation) and rapid cooling. Since the DNA is of high complexity, the localized concentration of the complementary sequence for any fragment may be negligible, thus allowing sufficient time to perform subsequent procedures when the DNA is mostly in the single stranded state. The use of ssDNA significantly simplifies circle formation because of the distinct polarity of 5′ and 3′ ends of each ssDNA fragment. The first stage is ligation of adaptor sequences to the ends of each single stranded genomic fragment. Since all possible sequence combinations may be represented in the genomic DNA, an adaptor can be ligated to one end with the aid of a bridging template molecule that is synthesized with all possible sequences. Since these oligonucleotides may be of relatively high concentration compared to the genomic DNA, the oligonucleotide that is complementary to the end of the genomic fragment (or a complement with mismatches) may hybridize. A bridge is thus formed at the ligation site to allow ligation of the 5-prime end of the single stranded genomic fragment to the adaptor.

FIG. 1E illustrates one method of attaching sequence tags and circularizing tag-template conjugates. Target nucleic acid (1600) is treated (1601) to form single stranded fragments (1602), for example, in the range of from 50 to 600 nucleotides, and preferably in the range of from 300 to 600 nucleotides, which are then ligated to sequence tag-containing adaptor oligonucleotides (1604) to form a population of adaptor-fragment conjugates (1606). Target nucleic acid (1600) may be genomic DNA extracted from a sample using conventional techniques, or a cDNA or genomic library produced by conventional techniques, or synthetic DNA, or the like. Treatment (1601) usually entails fragmentation by a conventional technique, such as chemical fragmentation, enzymatic fragmentation, or mechanical fragmentation, followed by denaturation to produce single stranded DNA fragments.

In generating target nucleic acids, fragments making up the target nucleic acids may be derived from either an entire genome or from a selected subset of a genome. Many techniques are available for isolating or enriching fragments from a subset of a genome, as exemplified by the following references, which are incorporated in their entirety by reference: Kandpal et al (1990), Nucleic Acids Research, 18: 1789-1795; Callow et al, U.S. patent publication 2005/0019776; Zabeau et al, U.S. Pat. No. 6,045,994; Deugau et al, U.S. Pat. No. 5,508,169; Sibson, U.S. Pat. No. 5,728,524; Guilfoyle et al, U.S. Pat. No. 5,994,068; Jones et al, U.S. patent publication 2005/0142577; Gullberg et al, U.S. patent publication 2005/0037356; Matsuzaki et al, U.S. patent publication 2004/0067493; and the like.

As will be appreciated by those in the art, there are several ways to form circularized adaptor/target sequence components. In one embodiment, a CircLigase™ enzyme is used to close single stranded polynucleotide circles without template. Alternatively, a bridging template that is complementary to the two termini of the linear strand is used. In some embodiments, the addition of a first adaptor to one termini of the target sequence is used to design a complementary part of the bridging template. The other end may be universal template DNA containing degenerate bases for binding to all genomic sequences. Hybridization of the two termini followed by ligation results in a circularized component. Alternatively, the 3′ end of the target molecule may be modified by addition of a poly-dA tail using terminal transferase. The modified target is then circularized using a bridging template complementary to the adaptor and to the oligo-dA tail.

In one method of circularization, illustrated in FIG. 2, after genomic DNA (200) is fragmented and denatured (202), single stranded DNA fragments (204) are first treated with a terminal transferase (206) to attach a poly dA tails (208) to 3-prime ends. This is then followed by ligation (212) of the free ends intra-molecularly with the aid of bridging oligonucleotide (210) that is complementary to the poly dA tail at one end and complementary to any sequence at the other end by virtue of a segment of degenerate nucleotides. Duplex region (214) of bridging oligonucleotide (210) contains at least a primer binding site for RCR and, in some embodiments, sequences that provide complements to a capture oligonucleotide, which may be the same or different from the primer binding site sequence, or which may overlap the primer binding site sequence. The length of capture oligonucleotides may vary widely, In one aspect, capture oligonucleotides and their complements in a bridging oligonucleotide have lengths in the range of from 10 to 100 nucleotides; and more preferably, in the range of from 10 to 40 nucleotides. In some embodiments, duplex region (214) may contain additional elements, such as an oligonucleotide tag, for example, for identifying the source nucleic acid from which its associated DNA fragment came. That is, in some embodiments, circles or adaptor ligation or concatemers from different source nucleic acids may be prepared separately during which a bridging adaptor containing a unique tag is used, after which they are mixed for concatemer preparation or application to a surface to produce a random array. The associated fragments may be identified on such a random array by hybridizing a labeled tag complement to its corresponding tag sequences in the concatemers, or by sequencing the entire adaptor or the tag region of the adaptor. Circular products (218) may be conveniently isolated by a conventional purification column, digestion of non-circular DNA by one or more appropriate exonucleases, or both.

DNA fragments of the desired sized range, e.g. 50-600 nucleotides, may be circularized using circularizing enzymes, such as CircLigase, as single stranded DNA ligase that circularizes single stranded DNA without the need of a template. A preferred protocol for forming single stranded DNA circles comprising a DNA fragment and one or more adaptors is to use a standard ligase, such as T4 ligase, for ligating an adaptor to one end of a DNA fragment followed by application of CircLigase to close the circle.

In some embodiments, RCA amplicons, comprising concatemers of sequence tag-template conjugates, are produced in a conventional rolling circle replication (RCR) reaction. Guidance for selecting conditions and reagents for RCA reactions is available in many references available to those of ordinary skill, as evidence by the following that are incorporated by reference: Fire et al, U.S. Pat. No. 5,648,245; Kool, U.S. Pat. No. 5,426,180; Lizardi, U.S. Pat. Nos. 5,854,033 and 6,143,495; Landegren, U.S. Pat. No. 5,871,921; and the like. Generally, RCA reaction components comprise single stranded DNA circles, one or more primers that anneal to DNA circles, a DNA polymerase having strand displacement activity to extend the 3′ ends of primers annealed to DNA circles, nucleoside triphosphates, and a conventional polymerase reaction buffer. Such components are combined under conditions that permit primers to anneal to DNA circles and be extended by the DNA polymerase to form concatemers of DNA circle complements. An exemplary RCA reaction protocol is as follows: In a 50 μL reaction mixture, the following ingredients are assembled: 2-50 pmol circular DNA, 0.5 units/μL phage φ29 DNA polymerase, 0.2 μg/μL BSA, 3 mM dNTP, 1×φ29 DNA polymerase reaction buffer (Amersham). The RCA reaction is carried out at 30° C. for 12 hours. In some embodiments, the concentration of circular DNA in the polymerase reaction may be selected to be low (approximately 10-100 billion circles per ml, or 10-100 circles per picoliter) to avoid entanglement and other intermolecular interactions.

### Samples

Samples (sometimes referred to as “tissue samples”) from which target nucleic acids are obtained can come from a variety of tissues, including, for example, tumor tissue, blood and blood plasma, lymph fluid, cerebrospinal fluid surrounding the brain and the spinal cord, synovial fluid surrounding bone joints, and the like. In one embodiment, the sample is a blood sample. The blood sample can be about 0.1, 0.2, 0.3, 0.4, 0.5, 0.6, 0.7, 0.8, 0.9, 1.0, 1.5, 2.0, 2.5, 3.0, 3.5, 4.0, 4.5, or 5.0 mL. The sample can be a tumor biopsy. The biopsy can be from, for example, from a tumor of the brain, liver, lung, heart, colon, kidney, or bone marrow. Any biopsy technique used by those skilled in the art can be used for isolating a sample from a subject. For example, a biopsy can be an open biopsy, in which general anesthesia is used. The biopsy can be a closed biopsy, in which a smaller cut is made than in an open biopsy. The biopsy can be a core or incisional biopsy, in which part of the tissue is removed. The biopsy can be an excisional biopsy, in which attempts to remove an entire lesion are made. The biopsy can be a fine needle aspiration biopsy, in which a sample of tissue or fluid is removed with a needle.

A sample or tissue sample includes nucleic acid, for example, DNA (e.g., genomic DNA) or RNA (e.g., messenger RNA). The nucleic acid can be cell-free DNA or RNA, e.g. extracted from the circulatory system, Vlassov et al, Curr. Mol. Med., 10: 142-165 (2010); Swamp et al, FEBS Lett., 581: 795-799 (2007). In the methods of the invention, the amount of RNA or DNA from a subject that can be analyzed includes varies widely. RNA used in methods of the invention may be either total RNA extracted from a tissue sample or polyA RNA extracted directly from a tissue sample or from total RNA extracted from a tissue sample. The above nucleic acid extractions may be carried out using commercially available kits, e.g. from Invitrogen (Carlsbad, Calif.), Qiagen (San Diego, Calif.), or like vendors. Guidance for extracting RNA is found in Liedtke et al, PCR Methods and Applications, 4: 185-187 (1994); and like references.

Blood samples are of particular interest and may be obtained using conventional techniques, e.g. Innis et al, editors, PCR Protocols (Academic Press, 1990); or the like. For example, white blood cells may be separated from blood samples using convention techniques, e.g. RosetteSep kit (Stem Cell Technologies, Vancouver, Canada). Likewise, other fractions of whole blood, such as peripheral blood mononuclear cells (PBMCs) may be isolated for use with methods of the invention using commercially available kits, e.g. Miltenyi Biotec, Auburn, Calif.), or the like. Blood samples may range in volume from 100 μL to 10 mL; in one aspect, blood sample volumes are in the range of from 200 100 μL to 2 mL. DNA and/or RNA may then be extracted from such blood sample using conventional techniques for use in methods of the invention, e.g. DNeasy Blood & Tissue Kit (Qiagen, Valencia, Calif.). Optionally, subsets of white blood cells, e.g. lymphocytes, may be further isolated using conventional techniques, e.g. fluorescently activated cell sorting (FACS)(Becton Dickinson, San Jose, Calif.), magnetically activated cell sorting (MACS)(Miltenyi Biotec, Auburn, Calif.), or the like.

### Sequencing Populations of Tag-Template Conjugates

Any high-throughput technique for sequencing nucleic acids can be used in the method of the invention. DNA sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, sequencing by synthesis, real time monitoring of the incorporation of labeled nucleotides during a polymerization step, polony sequencing, SOLiD sequencing, and the like. In some embodiments of the invention, high-throughput methods of sequencing are employed that comprise a step of spatially isolating individual molecules on a solid surface where they are sequenced in parallel. Such solid surfaces may include nonporous surfaces (such as in Solexa sequencing, e.g. Bentley et al, Nature, 456: 53-59 (2008) or Complete Genomics sequencing, e.g. Drmanac et al, Science, 327: 78-81 (2010)), arrays of wells, which may include bead- or particle-bound templates (such as with 454, e.g. Margulies et al, Nature, 437: 376-380 (2005) or Ion Torrent sequencing, U.S. patent publication 2010/0137143 or 2010/0304982), micromachined membranes (such as with SMRT sequencing, e.g. Eid et al, Science, 323: 133-138 (2009)), or bead arrays (as with SOLiD sequencing or polony sequencing, e.g. Kim et al, Science, 316: 1481-1414 (2007)). In some embodiments, such methods comprise amplifying the isolated molecules either before or after they are spatially isolated on a solid surface. Prior amplification may comprise emulsion-based amplification, such as emulsion PCR, or rolling circle amplification. Of particular interest is Solexa-based sequencing where individual template molecules are spatially isolated on a solid surface, after which they are amplified in parallel by bridge PCR to form separate clonal populations, or clusters, and then sequenced, as described in Bentley et al (cited above) and in manufacturer's instructions (e.g. TruSeq™ Sample Preparation Kit and Data Sheet, Illumina, Inc., San Diego, Calif., 2010); and further in the following references: U.S. Pat. Nos. 6,090,592; 6,300,070; 7,115,400; and EP0972081B1; which are incorporated by reference. In one embodiment, individual molecules disposed and amplified on a solid surface form clusters in a density of at least 10⁵ clusters per cm²; or in a density of at least 5×10⁵ per cm²; or in a density of at least 10⁶ clusters per cm².

The sequencing technique used in the methods of the provided invention can generate sequence reads of about 30 nucleotides, about 40 nucleotides, about 50 nucleotides, about 60 nucleotides, about 70 nucleotides, about 80 nucleotides, about 90 nucleotides, about 100 nucleotides, about 110, about 120 nucleotides per read, about 150 nucleotides, about 200 nucleotides, about 250 nucleotides, about 300 nucleotides, about 350 nucleotides, about 400 nucleotides, about 450 nucleotides, about 500 nucleotides, about 550 nucleotides, or about 600 nucleotides per read.

While the present invention has been described with reference to several particular example embodiments, those skilled in the art will recognize that many changes may be made thereto without departing from the spirit and scope of the present invention. The present invention is applicable to a variety of sensor implementations and other subject matter, in addition to those discussed above.

### DEFINITIONS

Unless otherwise specifically defined herein, terms and symbols of nucleic acid chemistry, biochemistry, genetics, and molecular biology used herein follow those of standard treatises and texts in the field, e.g. Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Abbas et al, Cellular and Molecular Immunology, 6^{th} edition (Saunders, 2007).

“Amplicon” means the product of a polynucleotide amplification reaction; that is, a clonal population of polynucleotides, which may be single stranded or double stranded, which are replicated from one or more starting sequences. The one or more starting sequences may be one or more copies of the same sequence, or they may be a mixture of different sequences. Preferably, amplicons are formed by the amplification of a single starting sequence. Amplicons may be produced by a variety of amplification reactions whose products comprise replicates of the one or more starting, or target, nucleic acids. In one aspect, amplification reactions producing amplicons are “template-driven” in that base pairing of reactants, either nucleotides or oligonucleotides, have complements in a template polynucleotide that are required for the creation of reaction products. In one aspect, template-driven reactions are primer extensions with a nucleic acid polymerase or oligonucleotide ligations with a nucleic acid ligase. Such reactions include, but are not limited to, polymerase chain reactions (PCRs), linear polymerase reactions, nucleic acid sequence-based amplification (NASBAs), rolling circle amplifications, and the like, disclosed in the following references that are incorporated herein by reference: Mullis et al, U.S. Pat. Nos. 4,683,195; 4,965,188; 4,683,202; 4,800,159 (PCR); Gelfand et al, U.S. Pat. No. 5,210,015 (real-time PCR with “taqman” probes); Wittwer et al, U.S. Pat. No. 6,174,670; Kacian et al, U.S. Pat. No. 5,399,491 (“NASBA”); Lizardi, U.S. Pat. No. 5,854,033; Aono et al, Japanese patent publ. JP 4-262799 (rolling circle amplification); and the like. In one aspect, amplicons of the invention are produced by PCRs. An amplification reaction may be a “real-time” amplification if a detection chemistry is available that permits a reaction product to be measured as the amplification reaction progresses, e.g. “real-time PCR” described below, or “real-time NASBA” as described in Leone et al, Nucleic Acids Research, 26: 2150-2155 (1998), and like references. As used herein, the term “amplifying” means performing an amplification reaction. A “reaction mixture” means a solution containing all the necessary reactants for performing a reaction, which may include, but not be limited to, buffering agents to maintain pH at a selected level during a reaction, salts, co-factors, scavengers, and the like.

“Fragment”, “segment”, or “DNA segment” refers to a portion of a larger DNA polynucleotide or DNA. A polynucleotide, for example, can be broken up, or fragmented into, a plurality of segments. Various methods of fragmenting nucleic acid are well known in the art. These methods may be, for example, either chemical or physical or enzymatic in nature. Enzymatic fragmentation may include partial degradation with a DNase; partial depurination with acid; the use of restriction enzymes; intron-encoded endonucleases; DNA-based cleavage methods, such as triplex and hybrid formation methods, that rely on the specific hybridization of a nucleic acid segment to localize a cleavage agent to a specific location in the nucleic acid molecule; or other enzymes or compounds which cleave DNA at known or unknown locations. Physical fragmentation methods may involve subjecting the DNA to a high shear rate. High shear rates may be produced, for example, by moving DNA through a chamber or channel with pits or spikes, or forcing the DNA sample through a restricted size flow passage, e.g., an aperture having a cross sectional dimension in the micron or submicron scale. Other physical methods include sonication and nebulization. Combinations of physical and chemical fragmentation methods may likewise be employed such as fragmentation by heat and ion-mediated hydrolysis. See for example, Sambrook et al., “Molecular Cloning: A Laboratory Manual,” 3rd Ed. Cold Spring Harbor Laboratory Press, Cold Spring Harbor, N. Y. (2001) (“Sambrook et al.) which is incorporated herein by reference for all purposes. These methods can be optimized to digest a nucleic acid into fragments of a selected size range.

“Kit” refers to any delivery system for delivering materials or reagents for carrying out a method of the invention. In the context of methods of the invention, such delivery systems include systems that allow for the storage, transport, or delivery of reaction reagents (e.g., primers, enzymes, internal standards, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. Such contents may be delivered to the intended recipient together or separately. For example, a first container may contain an enzyme for use in an assay, while a second container contains primers.

“Nucleic acid sequence-based amplification” or “NASBA” is an amplification reaction based on the simultaneous activity of a reverse transcriptase (usually avian myeloblastosis virus (AMV) reverse transcriptase), an RNase H, and an RNA polymerase (usually T7 RNA polymerase) that uses two oligonucleotide primers, and which under conventional conditions can amplify a target sequence by a factor in the range of 109 to 1012 in 90 to 120 minutes. In a NASBA reaction, nucleic acids are a template for the amplification reaction only if they are single stranded and contain a primer binding site. Because NASBA is isothermal (usually carried out at 41° C. with the above enzymes), specific amplification of single stranded RNA may be accomplished if denaturation of double stranded DNA is prevented in the sample preparation procedure. That is, it is possible to detect a single stranded RNA target in a double stranded DNA background without getting false positive results caused by complex genomic DNA, in contrast with other techniques, such as RT-PCR. By using fluorescent indicators compatible with the reaction, such as molecular beacons, NASBAs may be carried out with real-time detection of the amplicon. Molecular beacons are stem-and-loop-structured oligonucleotides with a fluorescent label at one end and a quencher at the other end, e.g. 5′-fluorescein and 3′-(4-(dimethylamino)phenyl)azo) benzoic acid (i.e., 3′-DABCYL), as disclosed by Tyagi and Kramer (cited above). An exemplary molecular beacon may have complementary stem strands of six nucleotides, e.g. 4 G's or C's and 2 A's or T's, and a target-specific loop of about 20 nucleotides, so that the molecular beacon can form a stable hybrid with a target sequence at reaction temperature, e.g. 41° C. A typical NASBA reaction mix is 80 mM Tris-HCl [pH 8.5], 24 mM MgCl2, 140 mM KCl, 1.0 mM DTT, 2.0 mM of each dNTP, 4.0 mM each of ATP, UTP and CTP, 3.0 mM GTP, and 1.0 mM ITP in 30% DMSO. Primer concentration is 0.1 μM and molecular beacon concentration is 40 nM. Enzyme mix is 375 sorbitol, 2.1 μg BSA, 0.08 U RNase H, 32 U T7 RNA polymerase, and 6.4 U AMV reverse transcriptase. A reaction may comprise 5 μL sample, 10 μL NASBA reaction mix, and 5 μL enzyme mix, for a total reaction volume of 20 μL. Further guidance for carrying out real-time NASBA reactions is disclosed in the following references that are incorporated by reference: Polstra et al, BMC Infectious Diseases, 2: 18 (2002); Leone et al, Nucleic Acids Research, 26: 2150-2155 (1998); Gulliksen et al, Anal. Chem., 76: 9-14 (2004); Weusten et al, Nucleic Acids Research, 30(6) e26 (2002); Deiman et al, Mol. Biotechnol., 20: 163-179 (2002). Nested NASBA reactions are carried out similarly to nested PCRs; namely, the amplicon of a first NASBA reaction becomes the sample for a second NASBA reaction using a new set of primers, at least one of which binds to an interior location of the first amplicon.

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g. exemplified by the references: McPherson et al, editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature >90° C., primers annealed at a temperature in the range 50-75° C., and primers extended at a temperature in the range 72-78° C. The term “PCR” encompasses derivative forms of the reaction, including but not limited to, RT-PCR, real-time PCR, nested PCR, quantitative PCR, multiplexed PCR, and the like. The particular format of PCR being employed is discernible by one skilled in the art from the context of an application. Reaction volumes range from a few hundred nanoliters, e.g. 200 nL, to a few hundred μL, e.g. 200 μL. “Reverse transcription PCR,” or “RT-PCR,” means a PCR that is preceded by a reverse transcription reaction that converts a target RNA to a complementary single stranded DNA, which is then amplified, e.g. Tecott et al, U.S. Pat. No. 5,168,038, which patent is incorporated herein by reference. “Real-time PCR” means a PCR for which the amount of reaction product, i.e. amplicon, is monitored as the reaction proceeds. There are many forms of real-time PCR that differ mainly in the detection chemistries used for monitoring the reaction product, e.g. Gelfand et al, U.S. Pat. No. 5,210,015 (“taqman”); Wittwer et al, U.S. Pat. Nos. 6,174,670 and 6,569,627 (intercalating dyes); Tyagi et al, U.S. Pat. No. 5,925,517 (molecular beacons); which patents are incorporated herein by reference. Detection chemistries for real-time PCR are reviewed in Mackay et al, Nucleic Acids Research, 30: 1292-1305 (2002), which is also incorporated herein by reference. “Nested PCR” means a two-stage PCR wherein the amplicon of a first PCR becomes the sample for a second PCR using a new set of primers, at least one of which binds to an interior location of the first amplicon. As used herein, “initial primers” in reference to a nested amplification reaction mean the primers used to generate a first amplicon, and “secondary primers” mean the one or more primers used to generate a second, or nested, amplicon. “Asymmetric PCR” means a PCR wherein one of the two primers employed is in great excess concentration so that the reaction is primarily a linear amplification in which one of the two strands of a target nucleic acid is preferentially copied. The excess concentration of asymmetric PCR primers may be expressed as a concentration ratio. Typical ratios are in the range of from 10 to 100. “Multiplexed PCR” means a PCR wherein multiple target sequences (or a single target sequence and one or more reference sequences) are simultaneously carried out in the same reaction mixture, e.g. Bernard et al, Anal. Biochem., 273: 221-228 (1999)(two-color real-time PCR). Usually, distinct sets of primers are employed for each sequence being amplified. Typically, the number of target sequences in a multiplex PCR is in the range of from 2 to 50, or from 2 to 40, or from 2 to 30. “Quantitative PCR” means a PCR designed to measure the abundance of one or more specific target sequences in a sample or specimen. Quantitative PCR includes both absolute quantitation and relative quantitation of such target sequences. Quantitative measurements are made using one or more reference sequences or internal standards that may be assayed separately or together with a target sequence. The reference sequence may be endogenous or exogenous to a sample or specimen, and in the latter case, may comprise one or more competitor templates. Typical endogenous reference sequences include segments of transcripts of the following genes: β-actin, GAPDH, β₂-microglobulin, ribosomal RNA, and the like. Techniques for quantitative PCR are well-known to those of ordinary skill in the art, as exemplified in the following references that are incorporated by reference: Freeman et al, Biotechniques, 26: 112-126 (1999); Becker-Andre et al, Nucleic Acids Research, 17: 9437-9447 (1989); Zimmerman et al, Biotechniques, 21: 268-279 (1996); Diviacco et al, Gene, 122: 3013-3020 (1992); Becker-Andre et al, Nucleic Acids Research, 17: 9437-9446 (1989); and the like.

“Primer” means an oligonucleotide, either natural or synthetic that is capable, upon forming a duplex with a polynucleotide template, of acting as a point of initiation of nucleic acid synthesis and being extended from its 3′ end along the template so that an extended duplex is formed. Extension of a primer is usually carried out with a nucleic acid polymerase, such as a DNA or RNA polymerase. The sequence of nucleotides added in the extension process is determined by the sequence of the template polynucleotide. Usually primers are extended by a DNA polymerase. Primers usually have a length in the range of from 14 to 40 nucleotides, or in the range of from 18 to 36 nucleotides. Primers are employed in a variety of nucleic amplification reactions, for example, linear amplification reactions using a single primer, or polymerase chain reactions, employing two or more primers. Guidance for selecting the lengths and sequences of primers for particular applications is well known to those of ordinary skill in the art, as evidenced by the following references that are incorporated by reference: Dieffenbach, editor, PCR Primer: A Laboratory Manual, 2^{nd} Edition (Cold Spring Harbor Press, New York, 2003).

“Quality score” means a measure of the probability that a base assignment at a particular sequence location is correct. A variety methods are well known to those of ordinary skill for calculating quality scores for particular circumstances, such as, for bases called as a result of different sequencing chemistries, detection systems, base-calling algorithms, and so on. Generally, quality score values are monotonically related to probabilities of correct base calling. For example, a quality score, or Q, of 10 may mean that there is a 90 percent chance that a base is called correctly, a Q of 20 may mean that there is a 99 percent chance that a base is called correctly, and so on. For some sequencing platforms, particularly those using sequencing-by-synthesis chemistries, average quality scores decrease as a function of sequence read length, so that quality scores at the beginning of a sequence read are higher than those at the end of a sequence read, such declines being due to phenomena such as incomplete extensions, carry forward extensions, loss of template, loss of polymerase, capping failures, deprotection failures, and the like.

“RCA,” or “rolling circle amplification,” means a process in which a primer is annealed to a circular DNA molecule and extended by a DNA polymerase in the presence of nucleoside triphosphates to produce an extension product that contains multiple copies of the complementary sequence of the circular DNA molecule.

“Sequence read” means a sequence of nucleotides determined from a sequence or stream of data generated by a sequencing technique, which determination is made, for example, by means of base-calling software associated with the technique, e.g. base-calling software from a commercial provider of a DNA sequencing platform. A sequence read usually includes quality scores for each nucleotide in the sequence. Typically, sequence reads are made by extending a primer along a template nucleic acid, e.g. with a DNA polymerase or a DNA ligase. Data is generated by recording signals, such as optical, chemical (e.g. pH change), or electrical signals, associated with such extension. Such initial data is converted into a sequence read.

“Sequence tag” (or “tag”) or “barcode” means an oligonucleotide that is attached to a polynucleotide or template molecule and is used to identify and/or track the polynucleotide or template in a reaction or a series of reactions. A sequence tag may be attached to the 3′- or 5′-end of a polynucleotide or template or it may be inserted into the interior of such polynucleotide or template to form a linear conjugate, sometime referred to herein as a “tagged polynucleotide,” or “tagged template,” or “tag-polynucleotide conjugate,” “tag-molecule conjugate,” or the like. Sequence tags may vary widely in size and compositions; the following references, which are incorporated herein by reference, provide guidance for selecting sets of sequence tags appropriate for particular embodiments: Brenner, U.S. Pat. No. 5,635,400; Brenner and Macevicz, U.S. Pat. No. 7,537,897; Brenner et al, Proc. Natl. Acad. Sci., 97: 1665-1670 (2000); Church et al, European patent publication 0 303 459; Shoemaker et al, Nature Genetics, 14: 450-456 (1996); Morris et al, European patent publication 0799897A1; Wallace, U.S. Pat. No. 5,981,179; and the like. Lengths and compositions of sequence tags can vary widely, and the selection of particular lengths and/or compositions depends on several factors including, without limitation, how tags are used to generate a readout, e.g. via a hybridization reaction or via an enzymatic reaction, such as sequencing; whether they are labeled, e.g. with a fluorescent dye or the like; the number of distinguishable oligonucleotide tags required to unambiguously identify a set of polynucleotides, and the like, and how different must tags of a set be in order to ensure reliable identification, e.g. freedom from cross hybridization or misidentification from sequencing errors. In one aspect, sequence tags can each have a length within a range of from 2 to 36 nucleotides, or from 4 to 30 nucleotides, or from 8 to 20 nucleotides, or from 6 to 10 nucleotides, respectively. In one aspect, sets of sequence tags are used wherein each sequence tag of a set has a unique nucleotide sequence that differs from that of every other tag of the same set by at least two bases; in another aspect, sets of sequence tags are used wherein the sequence of each tag of a set differs from that of every other tag of the same set by at least three bases.

What is claimed is:

1. A method for sequencing nucleic acids comprising:
preparing templates from nucleic acids in a sample;
labeling by sampling the templates to form tag-template conjugates, wherein substantially every template of a tag-template conjugate has a unique sequence tag;
linearly amplifying the tag-template conjugates;
generating a plurality of sequence reads from the linearly amplified tag-template conjugates; and
determining a nucleotide sequence of each of the nucleic acids based on the frequencies of each type of nucleotide at each nucleotide position of each plurality of sequence reads having identical sequence tags.

2. A method for determining a nucleotide sequence of a rare nucleic acid, the method comprising the steps of:
attaching sequence tags to nucleic acids from a sample to form tag-template conjugates, wherein substantially every nucleic acid of the tag-template conjugates has a unique sequence tag;
linearly amplifying the tag-template conjugates;
generating a plurality of sequence reads from the linearly amplified tag-template conjugates; and
determining a nucleotide sequence of each of the nucleic acids based on the frequencies of each type of nucleotide at each nucleotide position of each plurality of sequence reads having identical sequence tags.

3. A method for determining a nucleotide sequence of a rare nucleic acid, the method comprising the steps of:
attaching sequence tags to nucleic acids from a sample to form tag-template conjugates, wherein substantially every nucleic acid of the tag-template conjugates has a unique sequence tag;
linearly amplifying the tag-template conjugates so that an amplicon is formed comprising only copies or copies of copies of the tag-template;
generating a plurality of sequence reads for each copy of the tag-template conjugates in the amplicon; and
determining a nucleotide sequence of each of the nucleic acids based on the frequencies of each type of nucleotide at each nucleotide position of each plurality of sequence reads having identical sequence tags.

4. The method of claim 1 wherein said template or said nucleic acid is single stranded DNA.

5. The method of claim 1 wherein said step of generating a plurality of sequence reads comprises separately amplifying each of said tag-template conjugates and sequencing each of the separately amplified tag-template conjugates to provide said sequence reads.

6. The method of claim 5 wherein said step of separately amplifying is carried out h bridge PCR or emulsion PCR.

7. The method of claim 1 wherein said step of determining includes determining a plurality nucleotide at each nucleotide position of each plurality of sequence reads having identical sequence tags.

8. The method of claim 7 wherein said plurality nucleotide at each of said nucleotide positions is a majority of nucleotides at such position.

9. The method of claim 1 wherein said step of linearly amplifying is carried out by asymmetric PCR, NASBA or RCA.

10. The method of claim 2 wherein said template or said nucleic acid is single stranded DNA.

11. The method of claim 2 wherein said step of generating a plurality of sequence roads comprises separately amplifying each of said tag-template conjugates and sequencing each of the separately amplified tag-template conjugates to provide said sequence reads.

12. The method of claim 11 wherein said step of separately amplifying is carried out by bridge PCR or emulsion PCR.

13. The method of claim 2 wherein said step of determining includes determining a plurality nucleotide at each nucleotide position of each plurality of sequence reads having identical sequence tags.

14. The method of claim 13 wherein said plurality nucleotide at each of said nucleotide positions is a majority of nucleotides at such position.

15. The method of claim 2 wherein said step of linearly amplifying is carried out by asymmetric PCR, NASBA or RCA.

16. The method of claim 3 wherein said template or said nucleic acid is single stranded DNA.

17. The method of claim 3 wherein said step of generating a plurality of sequence reads comprises separately amplifying each of said tag-template conjugates and sequencing each of the separately amplified tag-template conjugates to provide said sequence reads.

18. The method of claim 17 wherein said step of separately amplifying is carried out by bridge PCR or emulsion PCR.

19. The method of claim 3 wherein said step of determining includes determining a plurality nucleotide at each nucleotide position of each plurality of sequence reads having identical sequence tags.

20. The method of claim 19 wherein said plurality nucleotide at each of said nucleotide positions is a majority of nucleotides at such position.


SEQUENCE LISTING (7 sequences)

SEQ ID NO: 1 - DNA, 24 bp, Artificial Sequence
  template
agttcgggctaacctgtagagcta

SEQ ID NO: 2 - DNA, 24 bp, Artificial Sequence
  template
agttcgggctaacctgtcgagcca

SEQ ID NO: 3 - DNA, 24 bp, Artificial Sequence
  template
agttctggctaacctgtagagcca

SEQ ID NO: 4 - DNA, 24 bp, Artificial Sequence
  template
agttctggctaacctgtagagcta

SEQ ID NO: 5 - DNA, 24 bp, Artificial Sequence
  template
agttctggctaacctgtagagcca

SEQ ID NO: 6 - DNA, 24 bp, Artificial Sequence
  template
agttcgggctaacctgtagagcca

SEQ ID NO: 7 - DNA, 24 bp, Artificial Sequence
  template
agttctggctaacttgtagagcca